SEQUENCE LISTING (7 sequences)

SEQ ID NO: 1 - protein, 39 aa, Homo sapiens
LSVATALSALSLGAEQRTESIIHRALYYDLISSPDIHGT

SEQ ID NO: 2 - protein, 34 aa, Homo sapiens
ALSALSLGAEQRTESIIHRALYYDLISSPDIHGT

SEQ ID NO: 3 - protein, 29 aa, Homo sapiens
SLGAEQRTESIIHRALYYDLISSPDIHGT

SEQ ID NO: 4 - protein, 24 aa, Homo sapiens
SLGAEQRTESIIHRALYYDLISSP

SEQ ID NO: 5 - protein, 20 aa, Homo sapiens
SLGAEQRTESIIHRALYYDL

SEQ ID NO: 6 - protein, 29 aa, Mus musculus
SLGAEHRTESVIHRALYYDLITNPDIHST

SEQ ID NO: 7 - protein, 20 aa, Mus musculus
SLGAEHRTESVIHRALYYDL

(19) United States
(12) Patent Application Publication
(10) Pub. No.: US 2026/0256881 A1
(43) Pub. Date: Sep. 3, 2026

(54) COMPOSITIONS COMPRISING PEDF-DERIVED SHORT PEPTIDES FOR THE TREATMENT OF NEUROTROPHIC KERATITIS DISEASES

(71) Applicant: BRIM Biotechnology, Inc., Taipei (TW)

(72) Inventors: Frank Wen-Chi LEE, Orlando, FL (US); Huei-Ling CHANG, Taipei (TW); Sin-Tzu NING, Taipei (TW); Chih-Te CHIEN, Taipei (TW); Ya-Lang HUANG, Taipei (TW)

(73) Assignee: BRIM Biotechnology, Inc., Taipei (TW)

(21) Appl. No.: 18/878,908

(22) PCT Filed: Jun. 26, 2023
(86) PCT No.: PCT/US2023/069086

§ 371 Date: Dec. 26, 2024

Related U.S. Application Data
(60) Provisional application No. 63/355,551, filed on Jun. 24, 2022.

(51) Int. Cl. A61K 38/17; A61P 27/02

(52) U.S. Cl. CPC A61K 38/179; A61P 27/02

(57) ABSTRACT

A method for treating neurotrophic keratitis (NK) includes administering to a subject in need thereof a composition that includes a peptide having the amino-acid sequence selected from SEQ ID NO: 1-7. The pharmaceutical composition contains about 10-200 μM PDSP. the pharmaceutical composition further comprising one or more other active ingredients.

### BACKGROUND OF THE INVENTION

The present invention relates to PEDF-derived short peptides (PDSPs) and their uses in the treatment and/or amelioration of neurotrophic keratitis disease.

Neurotrophic keratitis (NK) is a rare degenerative disease of the cornea caused by trigeminal nerve damage that results in the loss of corneal sensitivity, corneal epithelium breakdown, and poor corneal healing. NK was initially described as “neuroparalytic keratitis” by Magendie in 1824, who hypothesized the presence of trophic nerve fibers in the trigeminal nerve regulating tissue metabolism. Subsequently, it was demonstrated that the trigeminal nerve provides corneal sensation and supplies trophic factors to the cornea, playing a key role in maintaining corneal anatomical integrity and function of the ocular surface. Impairment of corneal trigeminal innervation causes morphological and metabolic epithelial disturbances and leads to development of recurrent or persistent epithelial defects. The impairment of corneal sensory innervation also causes the reduction of both protective reflexes and trophic neuromodulators that are essential for the vitality, metabolism, and wound healing of ocular surface tissues.

A wide range of ocular and systemic conditions, including herpetic keratitis, ocular chemical burns, corneal surgery, diabetes, multiple sclerosis, and neurosurgical procedures, can cause NK by damaging trigeminal innervation. As mentioned above, loss or reduction of corneal innervation leads to a reduced aqueous phase of the tear film and a consequential reduction in neurotransmitters/trophic factors that impair or inhibit epithelial healing capacity (impaired mitosis and migration). Combined existence of tear film deficiency and impaired epithelial healing capacity predispose affected individuals to persistent epithelial defects, corneal ulcers and perforation.

Early diagnosis, treatment and careful monitoring of neurotrophic keratitis patients are essential to achieve epithelial healing and prevent progression of corneal damage in NK patients. Management of NK is based on clinical severity and the aim of therapy is to halt the progression of corneal damage and promote epithelial healing. To assess severity, the clinical stages of NK are evaluated and range from corneal epithelial alterations (Stage 1) to persistent epithelial defect (Stage 2) and ulceration (Stage 3), which may progress to corneal perforation.

Clinical management of NK is clinical stage-dependent and can be broadly categorized as medical management, non-surgical intervention, and surgical management. Surgical treatments are reserved for refractory cases. Although several medical and surgical treatments have been proposed, no therapies are currently available to restore corneal sensitivity, and thus, NK remains difficult and challenging to treat. Only one topical treatment with nerve growth factor (NGF) been approved by FDA for the treatment of NK disease.

### SUMMARY OF THE INVENTION

One aspect of the invention relates to pharmaceutical compositions for treating neurotrophic keratitis (NK) in a subject. A pharmaceutical composition for treating NK in accordance with one embodiment of the invention comprises a peptide having the amino-acid sequence selected from SEQ ID NO: 1-7.

One aspect of the invention relates to methods for treating neurotrophic keratitis (NK) in a subject. A method for treating NK in accordance with one embodiment of the invention comprises administering to a subject in need thereof a composition comprising a peptide having the amino-acid sequence selected from SEQ ID NO: 1-7.

Other aspect of the invention will become apparent with the following description and the enclosed drawings.

### BRIEF DESCRIPTION OF DRAWINGS

FIG. 1 shows a schematic of treatment plan according to embodiments of the invention.

FIG. 2 shows treatment efficacies of a PDSP (SEQ ID NO:3; referred to as BRM424) with respect to corneal ulcer area. TA-1 group received BRM424 ophthalmic solution treatment, while TA-2 received vehicle treatment.

FIG. 3A and FIG. 3B show treatment efficacies of BRM424 with respect to corneal fluorescein staining score (FL score) and image of cornea surface, respectively.

FIG. 4A and FIG. 4B show treatment efficacies of BRM424 with respect to corneal sensitivity in Von Frey tests. FIG. 4A shows results of contact forces in terms of nylon sizes of Von Frey filaments that triggered corneal reflex. FIG. 4B shows results of contact forces in grams that triggered corneal reflex.

FIG. 5A shows treatment efficacies of BRM424 with respect to Schirmer Test.

FIG. 5B shows treatment efficacies of BRM424 with respect to Tear Film Break-up Time (TFBUT).

### DETAILED DESCRIPTION OF THE INVENTION

Human Pigment Epithelium-derived Factor (PEDF) is a secreted protein containing 418 amino acids, with a molecular weight of about 50 kDa. PEDF is a multifunctional protein with many biological functions (see e.g., U.S. Patent Application Publication No. 2010/0047212). Different peptide regions of the PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties.

U.S. Patent Application Publication No. 2010/0047212 discloses that PEDF can promote self-renewal of stem cells. U.S. Pat. Nos. 9,051,547 and 9,617,311 disclose that fragments of PEDF having 20-39 amino acids in length (residues 93-121 of PEDF) can promote stem cells proliferation and wound healing. These PEDF-derived short peptides are referred to as PDSP in this description. PDSP used in this invention are listed in TABLE 1 below:

TABLE 1

| Sequence | SEQ ID | Name | Residue No. |
|---|---|---|---|
| LSVATALSALSLGAEQRTESIIHRALYYDLISSPDIHGT | 1 | 39mer | 83-121 |
| ALSALSLGAEQRTESIIHRALYYDLISSPDIHGT | 2 | 34mer | 88-121 |
| SLGAEQRTESIIHRALYYDLISSPDIHGT | 3 | 29mer | 93-121 |
| SLGAEQRTESIIHRALYYDLISSP | 4 | 24mer | 93-116 |
| SLGAEQRTESIIHRALYYDL | 5 | 20mer | 93-112 |
| 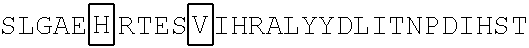 | 6 | mo29mer | |
| 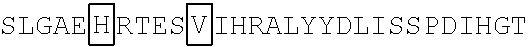 | 7 | mo20mer | |

Embodiments of the invention relate to PDSP and their uses in the prevention and/or treatment of neurotrophic keratitis (NK). NK is a rare degenerative ophthalmic disease with decreased corneal sensitivity, spontaneous destruction of corneal epithelial cells, and impairment of healing. In severe cases, NK can cause corneal ulceration, melting, and perforation, leading to impairment of the vision of patients. In preclinical studies, all PDSP listed in TABLE 1 have been found to be effective in preventing and/or treating NK. The following specific examples will use results from pre-clinical studies of the 29-mer (SEQ ID NO: 3, hereafter referred to as “BRM424”) to illustrate embodiments of the invention. One skilled in the art would appreciate that the results of the BRM424 are for illustration and are not intended to limit the scope of the invention.

The NK model is established in Chinchilla rabbits by using a chemical method to destroy the ophthalmic branch (V₁) of trigeminal nerve to induce NK. Briefly, animals were anesthetized. With careful blunt dissection, an opening was formed by pulling fascia between lateral rectus and inferior rectus on an inferior margin of the rabbit eye. The trigeminal nerve was carefully separated from surrounding fasciae, and the ophthalmic nerve (V₁) of trigeminal nerve was isolated and injured with sodium hydroxide solution. The V₁ injury was performed for unilateral eye in each animal for 5 days prior to treatment.

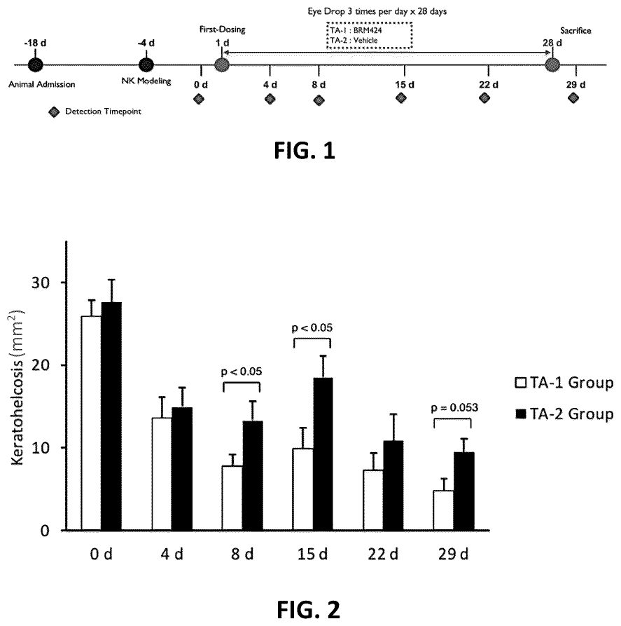

FIG. 1 shows a study schedule that included 28-day treatments. A total of 16 animals were assigned randomly into two groups based on corneal ulcer areas at Day 1 prior to the treatments, and the animals received either BRM424 ophthalmic solution (OS containing 0.03% BRM424; TA-1 group) or Vehicle (TA-2 group) in the surgical eye three times daily (TID, eye drops) for 28 days. During the study period, the corneal ulcer areas, corneal fluorescein staining, corneal sensitivity, Schirmer test, and Tear film break-up time (TFBUT) at baseline (Day 0), Day 4, Day 8, Day 15, Day 22, and Day 29 were measured.

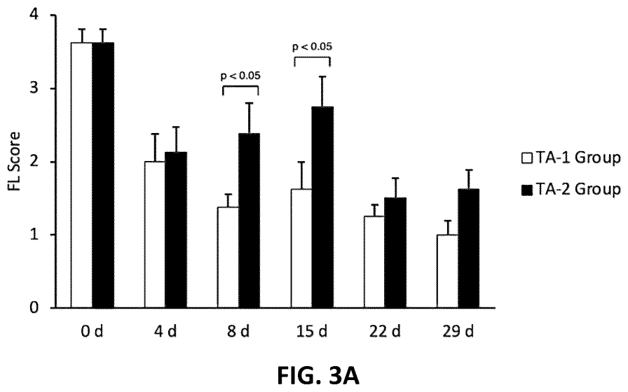

Corneal ulceration areas were measured from images taken during slit-lamp examination with cobalt blue light. FIG. 2 shows results for corneal ulceration area measurements. At Day 0, the corneal ulceration areas were 25.98±1.88 mm² for the TA-1 group and 27.65±2.66 mm² for the TA-2 group, and these areas were reduced to 7.80±1.45 mm² and 13.30±2.36 mm² by Day 8 for the TA-1 group and TA-2 group, respectively. The corneal ulceration area reductions of the TA-1 group were more than that of TA-2 group (p<0.05) at Day 8, Day 15, and Day 29, indicating that the TA-1 group had a rapid onset of action and was superior to the TA-2 group in promoting the corneal epithelial healing.

The results of corneal ulceration areas are summarized in TABLE 2.

TABLE 2
Corneal Ulceration Area (mm², X ± SEM)

| | | TA-1 BRM424 | TA-2 Vehicle | |
|---|---|---|---|---|
| | | (n = 8) | (n = 8) | p-value |
| 0 | d | 25.98 ± 1.88 | 27.65 ± 2.66 | NS |
| 4 | d | 13.63 ± 2.51 | 14.96 ± 2.28 | NS |
| 8 | d | 7.80 ± 1.45 | 13.30 ± 2.36 | 0.035 |
| 15 | d | 9.93 ± 2.46 | 18.50 ± 2.60 | 0.031 |
| 22 | d | 7.29 ± 2.11 | 10.90 ± 3.14 | NS |
| 29 | d | 4.81 ± 1.44 | 9.47 ± 1.67 | 0.053 |

NS: not significant

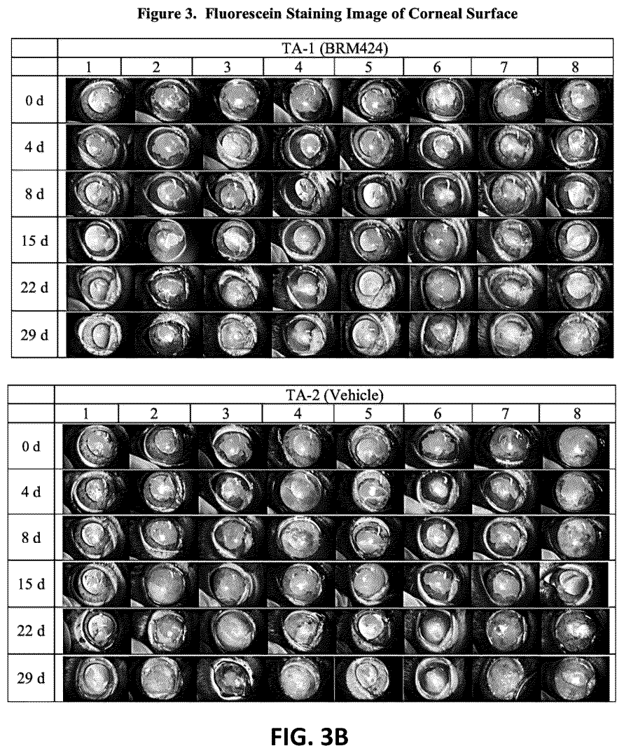

Cornea damage may be assessed using corneal fluorescein staining. FIG. 3A shows the results for corneal fluorescein staining (FL score), and FIG. 3B shows the staining images. The mean value of corneal fluorescein sodium staining scores at Day 0 was 3.62±0.18 for both the TA-1 and TA-2 groups and fell below 3 (TA-1=1.38±0.18, TA-2=2.36±0.42) by Day 8, indicating a quick onset of action. Significant difference between the TA-1 and TA-2 groups was observed at Day 8 and Day 15 (P<0.05). The fluorescein staining score of the TA-1 group reached 1 by Day 29.

The results of corneal area staining are summarized in TABLE 3.

TABLE 3
Fluorescein Staining Score (X ± SEM)

| | | | |
|---|---|---|---|
| | TA-1 BRM424 | TA-2 Vehicle | |
| Day | (n = 8) | (n = 8) | p-value |

| | | | | |
|---|---|---|---|---|
| 0 | d | 3.62 ± 0.18 | 3.62 ± 0.18 | NS |
| 4 | d | 2.00 ± 0.38 | 2.13 ± 0.35 | NS |
| 8 | d | 1.38 ± 0.18 | 2.38 ± 0.42 | 0.023 |
| 15 | d | 1.63 ± 0.36 | 2.75 ± 0.27 | 0.031 |
| 22 | d | 1.25 ± 0.16 | 1.50 ± 0.27 | NS |
| 29 | d | 1.00 ± 0.19 | 1.63 ± 0.26 | NS |

NS: not significant

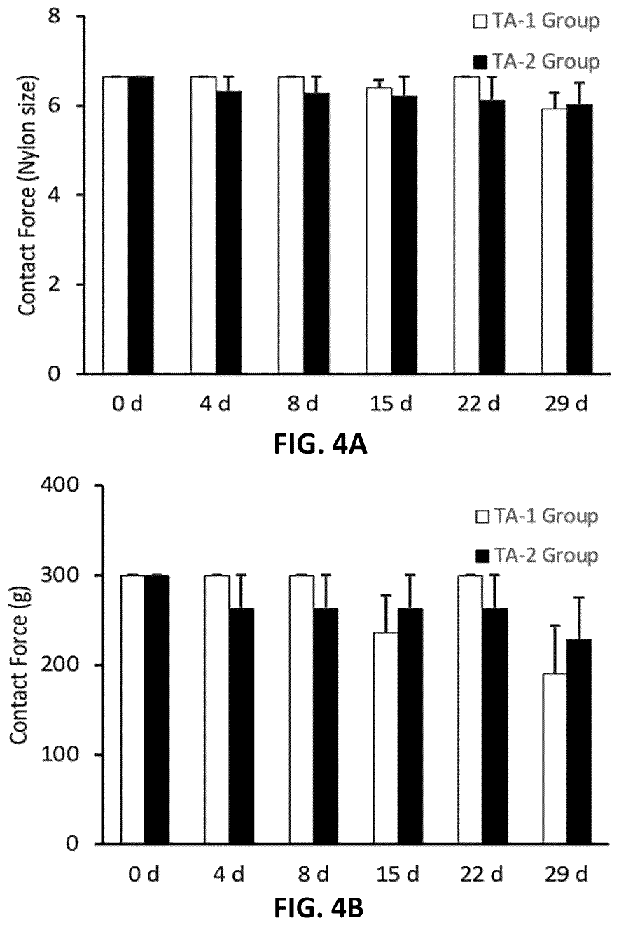

Central corneal sensitivity was monitored using Von Frey filaments. Various forces of calibrated Von Frey filaments (0.008-300 g) were applied to the center of the cornea surface of immobilized animal. The mechanical threshold response was determined by assessing treated-eye blinking response evoked by the same calibrated Von Frey filaments twice out of three times. FIG. 4A and FIG. 4B show results for corneal sensitivity tests. Prior to the dose administration (Day 0), there was no corneal reflex in all animals, even when using the coarsest nylon filament (Force 0.008-300 g). This result is consistent with the fact that the corneal sensory nerves are impaired.

As shown in FIG. 4B, after 29 days of treatments, the corneal sensation slowly recovered in both groups, while the corneal sensation of the TA-1 group recovered faster with better improvement than the TA-2 group. Corneal sensation improvement indicates the recovery of functional corneal sensory nerves. These results indicate that BRM424 can accelerate corneal sensory nerves recovery and, therefore, can be used to treat neurotrophic keratitis.

The results of corneal sensitivity tests are summarized in TABLE 4.

TABLE 4
Corneal Sensitivity (X ± SEM)

| | | Contact force (Nylon size) | | Contact force (g) | |
|---|---|---|---|---|---|
| | | TA-1BRM424 | TA-2 Vehicle | TA-1 BRM424 | TA-2 Vehicle |
| | | (n = 8) | (n = 8) | (n = 8) | (n = 8) |
| 0 | d | 6.65 ± 0.00 | 6.65 ± 0.00 | 300.00 ± 0.00 | 300.00 ± 0.00 |
| 4 | d | 6.65 ± 0.00 | 6.33 ± 0.32 | 300.00 ± 0.00 | 262.63 ± 37.38 |
| 8 | d | 6.65 ± 0.00 | 6.27 ± 0.38 | 300.00 ± 0.00 | 262.55 ± 37.45 |
| 15 | d | 6.41 ± 0.16 | 6.22 ± 0.43 | 235.75 ± 42.18 | 262.52 ± 37.48 |
| 22 | d | 6.65 ± 0.00 | 6.12 ± 0.52 | 300.00 ± 0.00 | 262.51 ± 37.50 |
| 29 | d | 5.93 ± 0.35 | 6.02 ± 0.48 | 189.75 ± 53.80 | 228.26 ± 47.03 |

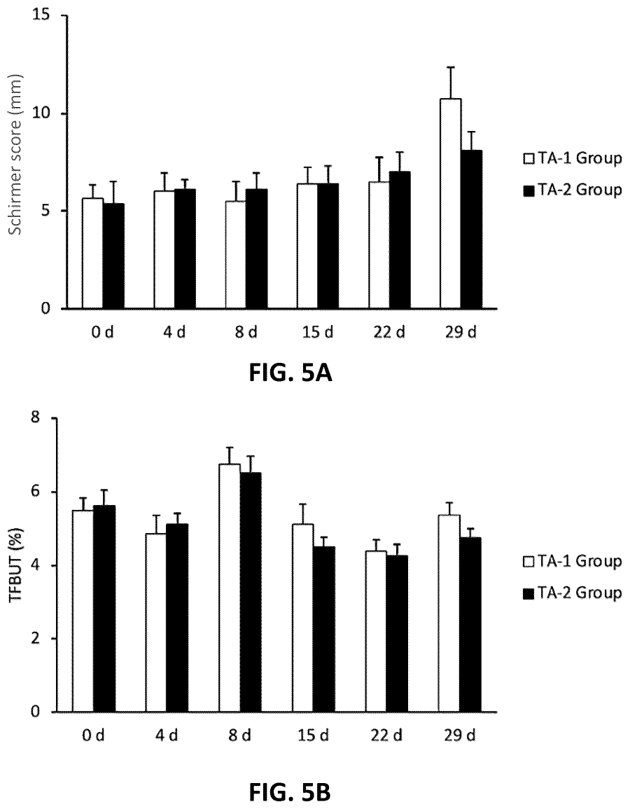

FIG. 5A shows the results for Schirmer Tests for tear productions. After 29 days of topical dose administrations, there was no significant difference in tear volumes between the TA-1 and TA-2 groups. However, the tear volumes of the TA-1 group at Day 29 were about 2-fold higher than that at Day 0. The Schirmer test results are summarized in TABLE 5.

TABLE 5
Schirmer Test (mm, X ± SEM)

| | | TA-1 BRM424 (n = 8) | TA-2 Vehicle (n = 8) |
|---|---|---|---|
| 0 | d | 5.63 ± 0.71 | 5.38 ± 1.13 |
| 4 | d | 6.00 ± 0.93 | 6.13 ± 0.48 |
| 8 | d | 5.50 ± 1.02 | 6.13 ± 0.81 |
| 15 | d | 6.38 ± 0.86 | 6.38 ± 0.94 |
| 22 | d | 6.50 ± 1.24 | 7.00 ± 1.02 |
| 29 | d | 10.75 ± 1.59 | 8.13 ± 0.95 |

FIG. 5B shows results of tear film break-up time studies and these results are summarized in TABLE 6. After 29 days of topical dose administrations, there was no significant difference between the TA-1 and TA-2 groups in tear film break-up time (TFBUT) (see FIG. 5B).

TABLE 6
TFBUT (second, X ± SEM)

| | | TA-1 BRM424 (n = 8) | TA-2 Vehicle (n = 8) |
|---|---|---|---|
| 0 | d | 5.50 ± 0.33 | 5.62 ± 0.42 |
| 4 | d | 4.88 ± 0.48 | 5.13 ± 0.30 |
| 8 | d | 6.75 ± 0.45 | 6.50 ± 0.46 |
| 15 | d | 5.13 ± 0.55 | 4.50 ± 0.27 |
| 22 | d | 4.38 ± 0.32 | 4.25 ± 0.31 |
| 29 | d | 5.38 ± 0.32 | 4.75 ± 0.25 |

The above results clearly demonstrate the efficacies of PDSP of the invention in the treatment of neurotrophic keratitis (NK). Embodiments of the invention relate to methods for preventing and/or treating NK in a subject. A subject in accordance with embodiments of the invention may be a human or an animal. A method in accordance with an embodiment of the invention may comprise administering to a subject in need of NK prevention or treatment with a composition comprising a peptide selected from any PDSP listed in TABLE 1. In accordance with examples of the invention, the compositions may comprise a peptide of the invention, or a salt of such a peptide, together with a pharmaceutically acceptable carrier or excipient, such as distill water, saline, oil, or gel.

A composition of the invention may be formulated in any suitable dosage forms, such as a solution, an ointment, a suspension, a gel or an emulsion, which may be formulated at any suitable concentrations, such as 10-200 μM. One skilled in the art would be able to formulate these at a suitable concentration to deliver an effective dose without inventive efforts. These dosage forms may be formulated for topical application to the eyes or other suitable routes of administrations (e.g., oral or injection).

While embodiments of the invention have been illustrated with a limited number of examples. One skilled in the art would appreciate that other modifications or variations are possible without departing from the scope of the invention. Therefore, the scope of protection should be limited by the accompanying claims.

1. A method for treating neurotrophic keratitis (NK), comprising: administering to a subject in need thereof a pharmaceutical composition comprising a peptide having the amino-acid sequence selected from SEQ ID NO: 1-7.

2. The method according to claim 1, wherein the peptide having the amino-acid sequence of SEQ ID NO: 3.

3. The method according to claim 1, wherein the pharmaceutical composition contains about 10-200 μM of the peptide.

4. The method according to claim 1, wherein the pharmaceutical composition further comprising one or more other active ingredients.

5. The method according to claim 2, wherein the pharmaceutical composition contains about 10-200 μM of the peptide.

6.-8. (canceled)
